(19) United States
(12) Patent Application Publication
(10) Pub. No.: US 2005/0000522 A1
(43) Pub. Date: Jan. 6, 2005

(54) Pad apparatus system for medical backboards

(76) Inventors: Garrett Henley Barnes, Rome, GA (US); Horace Lee Cline, Rome, GA (US); Todd L. Hold, Rome, GA (US)

Correspondence Address: RAJU LAW FIRM, LLC, P.O. BOX 12372, ATLANTA, GA, 30355, US

(21) Appl. No.: 10/613,644

(22) Filed: Jul. 3, 2003

(51) Int. Cl. A61F 5/37

(52) U.S. Cl. 128/870

(57) ABSTRACT

The present invention discloses a disposable pad apparatus for backboards. The pad apparatus releasably attaches to backboards using an adhesive mechanism. The pad apparatus is applied to the backboard prior to patient use and disposed of after patient use.

### BACKGROUND

(1) Field of the Invention

The present invention is related to medical equipment, and more particularly, to a disposable pad apparatus system for medical backboards.

(2) Description of the Background

Many field EMS/trauma situations require the use of spinal backboards for extrication, transportation, immobilization, and stabilization of patients with central or extremity bony injuries. Conventional backboards are rigid and substantially rectangular panels having a plurality of slots at proximate opposite side edges of the panel. The slots serve as handles for carrying the patient, and as anchors for straps that secure the patient to the board. Typically, the spine boards have an additional one pair or a pair of the slots proximate each end of the board.

Several problems arise when using backboards. A backboard is generally uncomfortable to a patient because the backboard is hard and rigid. This discomfort to a patient continues for the period a patient is immobilized. The patient is immobilized during the examination at the scene of the accident, ambulance transport, and examination at the hospital. Such extended immobilization may lead to additional trauma including soft tissue injury, skin breakdown, and decubitus ulcer formation. Also, backboards are significant conductors of thermal heat away from the patient causing negative clinical implications especially in the very young and elderly trauma patient. Thus there is a need for a material to be placed in between the patient and the backboard to provide cushion, comfort, protect the skin and soft-tissues of a patient and not conduct heat away from the body.

In the past blankets have been used to provide cushion, comfort and protection. However, blankets may slip off a backboard. Also, padding has been permanently fixed to a backboard. This, however, increases bulk and weight of a backboard thereby taking up valuable storage space.

There have been patents issued in the art of backboard padding:

U.S. Pat. No. 4,895,173, entitled “Spineboards”, issued to Richard Brault, et al on Jan. 23, 1990, discloses an invention related to a spineboard and parts thereof adapted for securement and transport of a person who may have suffered a spinal injury. The spineboard consists of a board having at least one longitudinal track adapted to receive in slidable connection at least one body harness and having means to secure a head harness; at least one body harness having a pad adapted to lie over the track and having at least one strapping means adapted to engage the track in slidable connection and to encircle and to secure a person to the board; and a head harness adapted to be located on the board.

U.S. Pat. No. 5,088,137, entitled “Backboard having removable pad”, issued to Eric R. Rose, on Feb. 18, 1992, discloses a backboard apparatus for supporting a patient. The apparatus includes a board having top and bottom surfaces, a plurality of side pairs of slots formed in an edge extremity of the board; and a pad assembly connected to the board including a pad having bottom and top surfaces bounded by sides and ends of the pad, and a first strap connected to the pad, having free ends extending beyond the sides of the pad and a fastener for connecting the ends to form a first closed loop, extending through a side pair of the slots and under the bottom surface of the board for securing the pad to the board. The apparatus can include a plurality of the first straps spaced apart on the pad. The board can include an additional pair of the slots located proximate end portions of the edge extremity, the apparatus including a pair of end straps connected to the pad for preventing longitudinal movement between the pad and the board. The pad can include a foam core and a cover, the core being formed of polyurethane or polyvinyl chloride. The pad assembly can be provided for use with a separate spine board.

U.S. Pat. No. 5,819,746, entitled “Removable spinal board padding”, issued to Ross T. Walton, on Oct. 13, 1998, discloses padding for a long spinal board. The padding consists of a polyurethane or polyvinyl chloride foam core that has a waterproof covering. The shape of the pad allows it to fit onto any adult size long spinal board currently on the market. Additional padding at one end of the board elevates the head of the patient to an anatomically neutral position. Slots within the padding running longitudinally and transversely permit straps to slide freely inside the padding. These straps secure the pad to the board and the patient to the pad.

U.S. Pat. No. 6,138,306, entitled “Backboard assembly with inflatable pad”, issued to Nabil L. Muhanna, on Oct. 31, 2000, discloses an inflatable pad for releasable attachment to a backboard to provide cushioned support to substantially an entire human body. The inflatable pad comprises a pad body having a length, a top end and a bottom end, a first side and a second side, and a selectively inflatable air retaining chamber integrated with said pad body. The inflatable pad includes an airtight interior for which a compressed air source is provided which selectively rapidly inflates the interior of the inflatable pad to provide cushioning of a person being carried upon the backboard without comprising the immobilization of the person. The inflatable pad is alternatively comprised of various sections specifically contoured to support the head, back, and legs of a person placed thereupon, and alternatively includes an integrated head restraining means.

The pads disclosed in the aforementioned patents generally fail to address many important issues arising in the bone injury situation requiring a backboard. In general, the padding described must be retained and stored. This is problematic as sanitary issues commonly arise in the healthcare area and especially in accident scenarios requiring the use of a backboard. Victims may extrude bodily liquids such as blood, sweat, vomit or tears, upon such non-disposable permanent padding. This can stain or de-sterilize the padding. Moreover, a person's bacteria, including odor causing bacteria, may grow on the padding increasing the risk of exposure to others to such organisms. To prevent these problems, the padding mentioned in the above patents must be washed and sterilized before being re-used. Moreover, such padding must be stored thereby taking up space in a hospital or ambulance.

In addition to the sanitary problems, the padding in the aforementioned patents can be time consuming to apply. Generally, they require the use of straps or some securing mechanism to keep the pads in place. Tying straps takes time and having to strap down the padding is an inconvenience. In an emergency situation time is of essence and an efficient application of padding to a backboard is required. The padding should not only save time by being easy to apply to the backboard but it should also be made of material that is inexpensive so it can be easily disposed.

Thus there remains a need of an invention that provides comfort, cushion, and bodily and thermal protection to patients, averts storage and contamination problems, and is efficient in terms of ease of and the amount of time for application of the invention to the backboard. The present invention addresses these needs.

### SUMMARY OF THE INVENTION

The present invention is directed to a disposable pad apparatus system for medical backboards.

Another embodiment of the invention is directed to a pad system apparatus of at least three sides for medical backboards, a right side, a left side, and a top side, and two surfaces, a top surface and a bottom surface, within which lies a cushion material.

Another embodiment of the invention is directed to a pad system apparatus of at least four sides for medical backboards, a right side, a left side, a top side, a bottom side, and two surfaces, a top surface and a bottom surface, between which lies a cushion material.

One embodiment of the present invention is directed to a pad apparatus system wherein the adhesive mechanism is a “peel and stick” adhesive tape. Another embodiment is directed to a pad apparatus system wherein the adhesive mechanism is VELCRO™.

One embodiment of the present invention is directed to an adhesive mechanism that has a pull tab such that facilitates the exposure of the sticky surface of the adhesive mechanism.

Another embodiment of the invention is directed to a pad system apparatus wherein the system is wrapped by a plastic material to keep the system in a sterile state.

Another embodiment of the invention is directed to methods of using the system.

Another embodiment is directed to a business method.

Other embodiments and advantages of the invention are set forth in the following description and, in part, will be obvious from this description, or may be learned from the practice of the invention.

### BRIEF DESCRIPTION OF DRAWINGS

For a fuller understanding of the nature of the present invention, reference should be had to the following detailed description taken in connection with the accompanying drawings in which:

FIG. 1 is a top view of a preferred embodiment of the pad apparatus system.

FIG. 2 is a bottom view of a preferred embodiment of the pad apparatus system.

FIG. 3 is a bottom view of a preferred embodiment of the pad apparatus system.

### DETAILED DESCRIPTION OF THE INVENTION

As shown in the accompanying drawings, the present invention relates to a disposable pad apparatus system for medical backboards, generally indicated by reference numeral 1, designed to be used in combination with a medical backboard which may have any number of sizes and configurations.

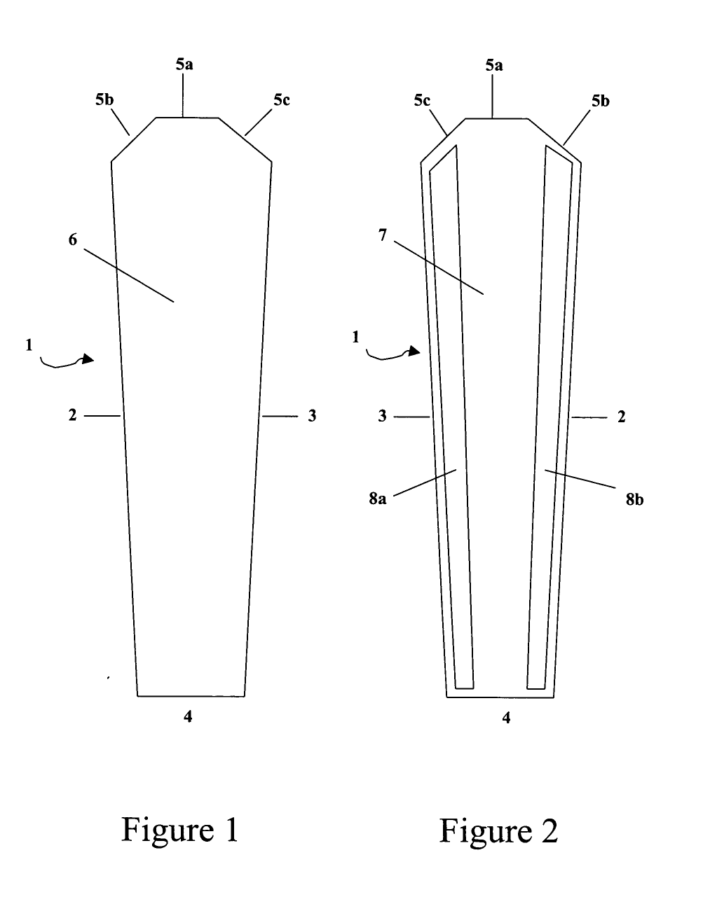

The invention provides a structure comprised of at least three sides, a right side, a left side, and a top side. FIGS. 1 and 2 illustrate one embodiment of the present invention, a pad apparatus system for medical backboards, 1, having six sides. As shown in FIGS. 1 and 2, in one preferred embodiment there is a right side, 2, a left side, 3, and a side, 4, a side 5a, a side 5b, and a side 5c.

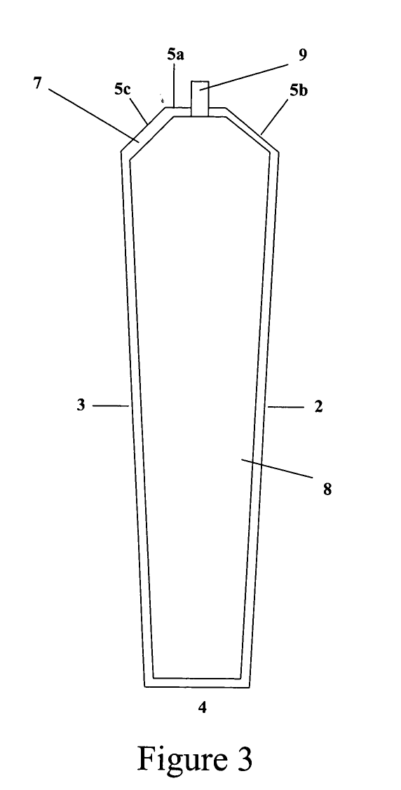

FIG. 1 is a top view of one embodiment of the pad apparatus system, 1. FIG. 2 is a bottom view of one embodiment of the pad apparatus system. In FIG. 1, a top surface, 6, is shown. In FIG. 2, a bottom surface, 7, is shown. The preferred embodiment of the invention has an adhesive mechanism attached to the bottom surface, 7, of the pad apparatus system, 1. In a more preferred embodiment, a plurality of adhesive mechanisms is attached to the bottom surface, 7, of the pad apparatus system, 1. As shown in FIG. 2, in one preferred embodiment two adhesive mechanisms, 8a and 8b respectively, align the left side, 3, and right side, 2, of the bottom side. These adhesive mechanisms are pre-attached to the bottom surface, 7, of the pad apparatus system, 1. The adhesive mechanisms may be of any size or shape. As shown in FIG. 3, in one preferred embodiment an adhesive mechanism, 8, substantially covers the bottom surface. In one preferred embodiment, the adhesive mechanism, 8, covers the entire bottom surface, 7.

In one preferred embodiment the adhesive mechanism is commonly known and used “peel and stick” adhesive tape. In an emergency situation the thin paper on adhesive tap is removed thereby exposing an adhesive, “sticky” surface, and the bottom surface, 7, of the pad apparatus system is thereupon placed on the appropriate side of a medical backboard, namely the side upon which a patient lies or will lie. A simple “peel and stick” design enables EMS providers to quickly attach the pad to any spine board, without interfering with the necessary conventional patient securing practices. i.e., straps, tape, etc. In another preferred embodiment the adhesive mechanism is a peel and stick VELCRO™ device. As shown in FIG. 3, in another preferred embodiment a pull tab, 9, is attached to the adhesive mechanism and facilitates the initial peeling process. A person will hold the tab, 9, and then peel off the thin paper that covers the adhesive mechanism.

In a preferred embodiment of the invention the padding of the pad apparatus system, 1, is made of polyurethane or polyvinyl chloride foam core. In a more preferred embodiment of the invention, the padding is waterproof. Such material is generally inexpensive and disposable. The preferred embodiment is a disposable padding apparatus system, 1. After the pad apparatus system, 1, has been put to use, it is simply pulled off the spine board and disposed thereby eliminating the need for costly and time consuming clean-up such as washing and de-sterilization. Single use application greatly reduces risk of exposure to potential pathogens to subsequent patients. In a preferred embodiment, the padding apparatus system, 1, comes wrapped in plastic and is sterilized. Thus, there is no worry of contamination of the padding.

In a preferred embodiment of the invention, the pad apparatus system, 1, is pre-sized. Pre-sized means the pad apparatus system, 1, is contoured to specific dimensions of a backboard. Preferably the padding apparatus system, 1, is pre-sized in many sizes such that may fit onto a conventional backboard. A “conventional backboard” is any adult or child size spinal board used in the market. In a more preferred embodiment of the invention, the dimensions of a pad according to the invention are such that it is adequately narrow to allow handholds on conventional adult spinal boards to be fully exposed. The end of the pad that will accommodate the head tapers when viewed from above to allow the use of the pad on boards that have similar tapering.

The right side, 2, and left side, 3, will be about the length of a backboard. “About the length” means the length of a conventional backboard but not so long as to cover the slots of a backboard or interfere with patient securing devices.

The top side of a preferred embodiment of the pad apparatus system, 1, will be “about the width” of the head end of a conventional backboard. The width on the top side will accommodate tapering of the backboard at the head end. The bottom side of a preferred embodiment of the pad apparatus system will be “about the width” of the foot end of a conventional backboard. “About the width” of a backboard means the width of a conventional backboard but not so wide as to cover the slots of a backboard or interfere with patient securing devices.

In a preferred embodiment of the invention, the padding apparatus system, 1, is uncut. “Uncut” means the pad apparatus system, 1, is in a state whereby it may be shaped by a person such as an emergency technician according to a shape dictated by such person using a shaping apparatus. A shaping apparatus includes scissors or any device that can adequately cut the padding to fit a backboard.

In a preferred embodiment of the invention, the padding apparatus system, 1, has print. In a more preferred embodiment of the invention, the print is instructions. In this embodiment, instructions for using the padding apparatus system, 1, are printed upon the padding apparatus system, 1, itself. In one embodiment, the instructions may be printed on the top surface, 6, of the padding apparatus system, 1. In another embodiment, the instructions may be printed on the bottom surface, 7, of the padding apparatus system, 1. In an even more preferred embodiment the instructions are printed on the top surface, 6, and bottom surface, 7, of the padding apparatus system, 1.

In another preferred embodiment of the invention, indicia or advertising displaying logos are printed on the padding apparatus system, 1.

In another preferred embodiment of the invention, a plastic material encloses the pad apparatus system, 1, such as to keep the pad apparatus system, 1, in a sterile state. The plastic material is wrapped around the padding. To use the padding the plastic material must first be unwrapped.

The present invention is directed also at methods of using the invention. A preferred embodiment of the invention is using the padding apparatus system, 1, by exposing the adhesive mechanism. Exposing the adhesive mechanism includes peeling off the paper of a “peel and stick” adhesive mechanism, applying the bottom surface, 7, of the pad apparatus system, 1, onto the side of a backboard on which is a patient is to lie, and finally disposing of the pad apparatus system, 1. Another preferred method of using the invention includes first unwrapping a plastic used to keep the padding apparatus system, 1, sterilized, then exposing the adhesive mechanism, then applying the bottom surface, 7, of the pad apparatus system, 1, onto the side of a backboard on which a patient is to lie, and then finally disposing of the pad apparatus system, 1.

After the pad apparatus system has been used, it is thereafter disposed. Disposal includes peeling off the pad from an associated backboard by simply grasping one end of the pad and peeling it off the backboard.

The present invention is also directed to a method of doing business. One preferred embodiment of the invention is selling the pad apparatus system, 1, as an accessory with a spinal backboard.

Other embodiments and uses of the invention will be apparent to those skilled in the art from consideration of the specification and practice of the invention disclosed herein. references cited herein are specifically and entirely hereby incorporated by reference. It is intended that the specification and examples be considered exemplary only. All modifications and improvements have been deleted herein for the sake of conciseness and readability but are properly within the scope of this provisional patent application.

1. A disposable pad apparatus system for an associated backboard comprising
a) at least three sides, the at least three sides further comprising at least
a right side having a length about the length between of the associated backboard,
a left side having a length about the length of the associated backboard and
a top side, having a width about the width of the associated backboard,
e) a top surface,
f) a bottom surface,
g) said bottom surface having an adhesive mechanism attached thereto, said adhesive mechanism used to releasably attach the pad apparatus system to the associated backboard, and
h) a cushion material between said top surface and said bottom surface.

2. The system according to claim 1 wherein the adhesive mechanism is “peel and stick” adhesive tape.

3. The system according to claim 1 wherein the adhesive mechanism is VELCRO™.

4. The system according to claim 1 wherein a pull tab is attached to the adhesive mechanism to facilitate peeling.

5. The system according to claim 1 wherein a plurality of adhesive mechanisms is attached to the bottom surface.

6. The system according to claim 1 wherein the cushion material is selected from the group consisting of polyurethane, polyvinyl chloride, and foam.

7. The system according to claim 1 wherein the system is wrapped by a plastic material to keep the pad apparatus system in a sterile state.

8. The system according to claim 1 wherein the top surface has print.

9. The system according to claim 1 wherein the bottom surface has print.

10. The system according to claim 1 wherein the disposable pad apparatus system is pre-sized.

11. The system according to claim 1 wherein the disposable pad apparatus system is uncut.

12. A disposable pad apparatus system for an associated backboard comprising
a) at least four sides, the at least four sides further comprising at least
a right side having a length about the length of the associated backboard,
a left side having a length about the length of the associated backboard,
a top side having a width about the width of the associated backboard and
a bottom side having a width about the width of the associated backboard
e) a top surface,
f) a bottom surface,
g) said bottom surface having an adhesive mechanism attached thereto, said adhesive mechanism used to releasably attach the pad apparatus system to the associated backboard and,
h) a cushion material between said top surface and said bottom surface.

13. The system according to claim 12 wherein the adhesive mechanism is “peel and stick” adhesive tape.

14. The system according to claim 12 wherein a pull tab is attached to the adhesive mechanism to facilitate peeling.

15. The system according to claim 12 wherein the adhesive mechanism is VELCRO™.

16. The system according to claim 12 wherein a plurality of adhesive mechanisms is attached to the bottom surface.

17. The system according to claim 12 wherein the cushion material is selected from the group consisting of polyurethane, polyvinyl chloride, and foam.

18. The system according to claim 12 wherein the system is wrapped by a plastic material to keep the pad apparatus system in a sterile state.

19. The system according to claim 12 wherein the top surface has print.

20. The system according to claim 12 wherein the bottom surface has print.

21. The system according to claim 12 wherein the disposable pad apparatus system is pre-sized.

22. The system according to claim 12 wherein the disposable pad apparatus system is uncut.

23. A disposable pad apparatus system for an associated backboard comprising
a) at least four sides, the at least four sides further comprising at least
a right side,
a left side,
and a top side,
and a bottom side
b) said right side having a length about the length of the associated backboard
c) said left side having a length about the length of the associated backboard
d) said top side having a width about the width of the associated backboard
e) said bottom side having a width about the width of the associated backboard
e) a top surface,
f) a bottom surface,
g) said bottom surface having “peel and stick” tape attached thereto, said “peel and stick” tape used to releasably attach the pad apparatus system to the associated backboard, and
h) a cushion material between said top surface and said bottom surface.

24. The system according to claim 23 wherein the adhesive mechanism is “peel and stick” adhesive tape.

25. The system according to claim 23 wherein a pull tab is attached to the adhesive mechanism to facilitate peeling.

26. The system according to claim 23 wherein the adhesive mechanism is VELCRO™.

27. The system according to claim 23 wherein a plurality of adhesive mechanisms is attached to the bottom surface.

28. The system according to claim 23 wherein the cushion material is selected from the group consisting of polyurethane, polyvinyl chloride, and foam.

29. The system according to claim 23 wherein the system is wrapped by a plastic material to keep the pad apparatus system in a sterile state.

30. The system according to claim 23 wherein the top surface has print.

31. The system according to claim 23 wherein the bottom surface has print.

32. The system according to claim 23 wherein the disposable pad apparatus system is pre-sized.

33. The system according to claim 23 wherein the disposable pad apparatus system is uncut.

34. A method of using the pad apparatus system having
at least three sides, the at least three sides further comprising at least
a right side having a length about the length between of the associated backboard,
a left side having a length about the length of the associated backboard and
a top side, having a width about the width of the associated backboard,
a top surface,
a bottom surface,
said bottom surface having an adhesive mechanism attached thereto, said
adhesive mechanism used to releasably attach the pad apparatus system to the associated backboard, and
a cushion material between said top surface and said bottom surface,
comprising the steps of
a)exposing the adhesive mechanism
b) setting the pad apparatus system upon the associated backboard by placing the bottom surface on the associated backboard and
c) disposing the pad apparatus system.

35. A method of using the pad apparatus system having
at least three sides, the at least three sides further comprising at least
a right side having a length about the length between of the associated backboard,
a left side having a length about the length of the associated backboard and
a top side, having a width about the width of the associated backboard,
a top surface,
a bottom surface,
said bottom surface having an adhesive mechanism attached thereto, said
adhesive mechanism used to releasably attach the pad apparatus system to the associated backboard, and
a cushion material between said top surface and said bottom surface,
said system wrapped by a plastic material to keep the pad apparatus system in a sterile state,
comprising the steps of
a) unwrapping a plastic used to keep the padding system apparatus sterilized
b) exposing the adhesive mechanism, and
c) setting the pad apparatus system upon the associated backboard by placing the bottom surface on the associated backboard and
d) disposing the pad apparatus system.

36. A method of doing business comprising selling a padding apparatus system in conjunction with an associated backboard.
